(19) United States
(12) Patent Application Publication
(10) Pub. No.: US 2008/0228188 A1
(43) Pub. Date: Sep. 18, 2008

(54) SURGICAL GUIDE

(76) Inventors: Alec Paul Birkbeck, Leeds (GB); Gary Moore, West Yorkshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/859,489

(22) Filed: Sep. 21, 2007

(30) Foreign Application Priority Data
Mar. 22, 2005 (GB) 0505782.3
Mar. 21, 2006 (GB) PCT/GB2006/001006

(51) Int. Cl. A61B 17/90

(52) U.S. Cl. 606/87

(57) ABSTRACT

A surgical guide and computer aided surgery method of use thereof are described. The guide is mounted on a body part to guide a device along a guide axis. The body has a channel in it which defines the guide axis and which receives the device in use. A base engages the surface of the body part when mounted on it in use, and supports the body over an entry point for the body part with the guide axis passing through the entry point. A pivot mechanism attaches the body to the base. The body can move relative to the base by pivoting only. The guide is configured such that the entry point is a centre of motion about which the body pivots.

The present invention relates to a guide, and in particular to a surgical guide for use in determining an axis relative to a body part and which is particularly suitable for use in computer aided surgery procedures.

A guide can be used during a surgical procedure to help ensure that an instrument or tool is applied to a body part by a practitioner at a particular location and/or with a particular orientation. A guide can have mechanisms providing various degrees of freedom so that the guide can be adjusted in use. While this provides flexibility in use, it also makes the guide less easy to use in practice and increases the size and complexity of the guide. Further, it can be difficult to rigidly attach the guide to bone. Hence, it may not be possible to use the guide at confined surgical sites. Further, the adjustment mechanism may not be accessible by the practitioner in situ. Further more, the mechanical complexity of the guide provides more scope for mechanical failure.

Hence, it would be beneficial to provide a simple, adjustable surgical guide, and in particular a navigable guide suitable for use in computer aided surgery (“CAS”) procedures.

According to a first aspect of the present invention, there is provided a guide mountable on a body part to guide a device along a guide axis. The guide can comprise a body having a channel therein. The channel defines the guide axis and can receive the device in use. A base can engage a surface of the body part when mounted thereon in use. The base supports the body over an entry point for the body part so that the guide axis is directed at the entry point. A pivot mechanism connects the body to the base. The body can move relative to the base by pivoting only. The guide is configured such that body pivots about the entry point.

As the guide is configured so that the body pivots about the entry point, this causes the guide axis to always pass through the entry point and so by placing the guide over the entry point with the guide axis directed through the entry point, the body can be pivoted only so as to align the guide axis and the axis of the body part. Hence, there is no need for other adjustment mechanisms and so a guide device of simple construction can be provided.

The guide can be configured such that it has a centre of motion about which the body pivots and the centre of motion can be located substantially in a plane defined by a footing or surface engaging part of the base. Hence, when the base is located on the body part with the centre of motion coincident with the entry point, the body will pivot about the entry point.

The guide can be a surgical guide. The guide can be a surgical guide for use in an orthopaedic surgical procedure. The guide can be for use in an articular surface replacement procedure. The articular surface can be of a head of a femur.

The guide can further comprise a lock operable to fix the angular position of the body relative to the base. In this way the guide can be locked in position once it has been aligned so as to provide a robust support which resists movement under the load of any tools, instruments or devices being guided in use.

The pivot mechanism can include a ball and socket joint provided by a pivoting part and a stationary part. At least one of the pivoting part and the stationary part can have a bearing surface being at least a part of a sphere, and the other can have a bearing surface or bearing structure over which the spherical bearing surface can move. The bearing structure can be provided by at least three elements, for example three roller balls. The pivoting part can have a bearing surface being at least a part of a sphere, and the stationary part can have a bearing surface being at least a part of a sphere. The stationary ball part can be a part of the base. The pivoting part can be a distal part of the body. This provides a mechanism by which the base and body part are prevented from separating and are aligned.

The pivot mechanism can include an element having a first bearing surface being a part of the surface of a sphere. The pivot mechanism can include a second bearing surface over which the first bearing surface can move. The radius of curvature of the first bearing surface can extend between the first bearing surface and a centre of motion of the guide about which the body rotates. Hence the first bearing surface and second bearing surface help to constrain the motion of the body so that the guide axis always passes through the centre of motion.

The base can comprises a plurality of attachment points each for receiving a fixing for securing the guide to the body part. In this way the guide can be prevented from moving during use so that the centre of motion and entry pointy remain coincidental. The fixing can be a pin or screw or other similar fixing suitable for securely attaching the guide to bone. The attachment points can be equi-angularly positioned about a central longitudinal axis of the base. There can be at least three attachment points. The attachment points can be apertures in a skirt or flange part surrounding a lower part of the base. The attachment points can be surface engaging formations, such as pins or friction surfaces. The attachment points allow the device to be held in a position on the body part by the user.

The guide can include a stem. The stem can be attached to the body and can pass into the base. The stem can include a further channel being an extension of the channel and co-linear with the guide axis. The stem can be attached at a distal end of the body and can provide a robust guide to the device or instrument through the base so as to help ensure that the device is guided to follow the correct axis or direction.

The stem can be attached to the body and can pass into, and/or be retained by, the base. The body and stem can co-operate to provide the lock. In this way the stem can help prevent the body and base from disengaging and/or provide an integral locking mechanism so as to provide a simple guide design with a reduced number of parts.

The guide can further include a marker attached to the body. The marker can be trackable by a tracking system to determine the position of the guide axis in a co-ordinate system of the tracking system. In this way a navigable guide adapted for use in a computer aided surgery system can be provided. The marker can be a wire based or a wireless marker. The marker can emit, transmit, reflect, or otherwise send energy detectable by a tracking system. The marker can send energy in acoustic or electromagnetic forms. The marker can be an ultrasonic marker, an infra red marker, an electromagnetic marker, a magnetic marker or an RF marker.

Each part of the guide can be rotationally symmetric about its longitudinal axis. Hence, the user does not need to carefully aligned the angular orientation of the guide when mounting it on a body part as the components are rotationally symmetric about the centre of motion and longitudinal axis of the guide which passes therethrough. Further this makes the components of the guide easy to manufacture.

According to a further aspect of the invention, there is provided a method for determining an axis along which a device is to be directed toward a body part. The method can comprise mounting a guide on the body part over an entry point on the surface of the body part with a centre of motion of the guide about which a guide axis can be pivoted only substantially co-incident with the entry point. A body of the guide having a channel therein defining the guide axis can be moved to vary the orientation of the guide axis while passing through the entry point until the guide axis is substantially coincidental with the axis.

Hence, as the guide is mounted with the centre of motion about which the guide axis can be pivoted coincident with the entry point, and as the guide axis can be pivoted only, the guide axis and axis can be aligned by pivoting the body of the guide only. Hence, a simpler method for determining an axis along which a device is to be guided is provided.

According to a further aspect of the invention, there is provided a CAS method for determining an axis along which a device is to be directed toward a body part. The method can comprise determining the orientation of the axis and an entry point through which the axis passes on the surface of a body part in a co-ordinate system of a tracking system. The position of a guide in the co-ordinate system can be tracked. The guide has a base and a body part, the body part having a channel for receiving the device along a guide axis and being movable relative to the base by pivoting only. A graphical indication of the position of the guide axis relative to the axis can be displayed. A graphical indication of the position of the guide axis relative to the entry point can also be displayed.

Hence, the method can be used to simplify an IGS part of a surgical procedure. The CAS system can guide the user to accurately position the guide on the body part relative to a determined entry point and once in position, only the angular position of the guide can be displayed in order to provide visual feedback allowing the user to assess the alignment of the guide axis and planned axis. This two stage process is easier for the user than trying to control a four degree of freedom positioning of the guide in a single step.

The graphical indication can include a graphical indication of the position of the guide axis and the axis. The graphical indication can include an indication of the current difference between the guide axis and the axis.

According to a further aspect of the invention, there is provided computer program code executable by a data processing device to provide the method aspect of the invention or to provide a CAS system aspect of the invention. A computer program product comprising a computer readable medium bearing such computer program code is also provided as an aspect of the invention.

An embodiment of the invention will now be described, by way of example only, and with reference to the accompanying drawings, in which:

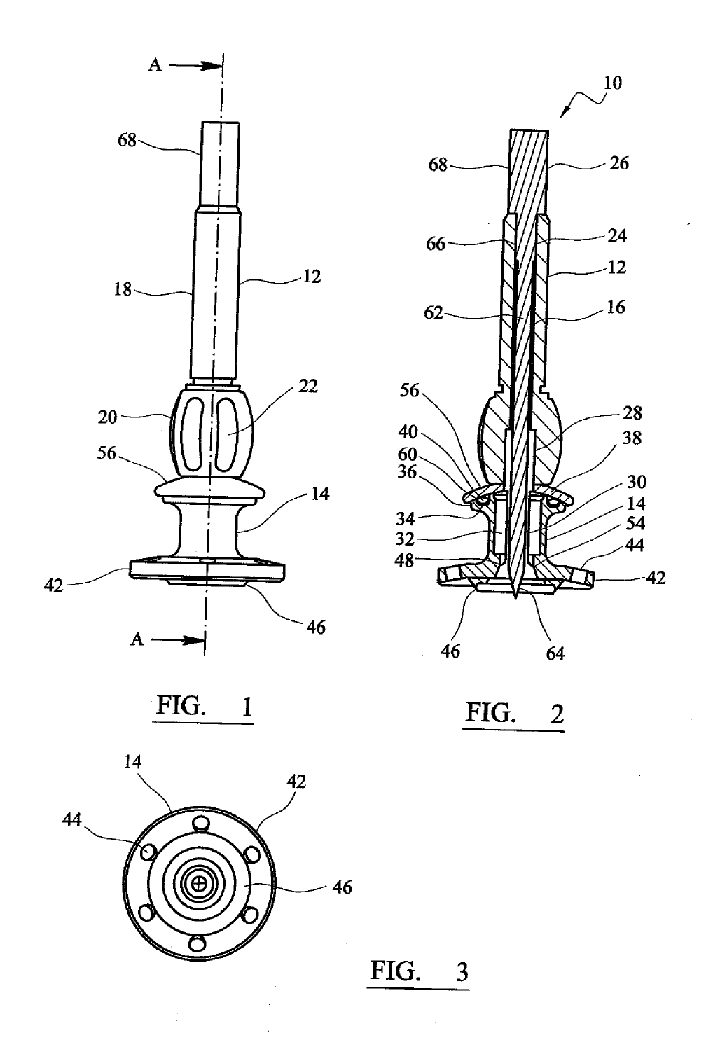

FIG. 1 shows a side view of a surgical guide according to the invention;

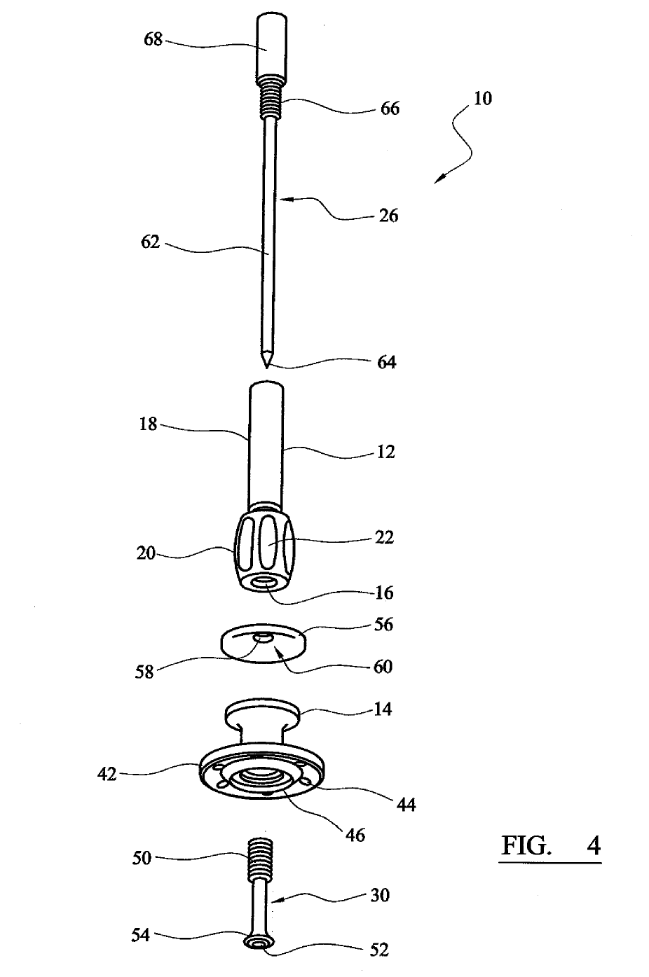

FIG. 2 shows a cross sectional view along line AA′ of the guide shown in FIG. 1;

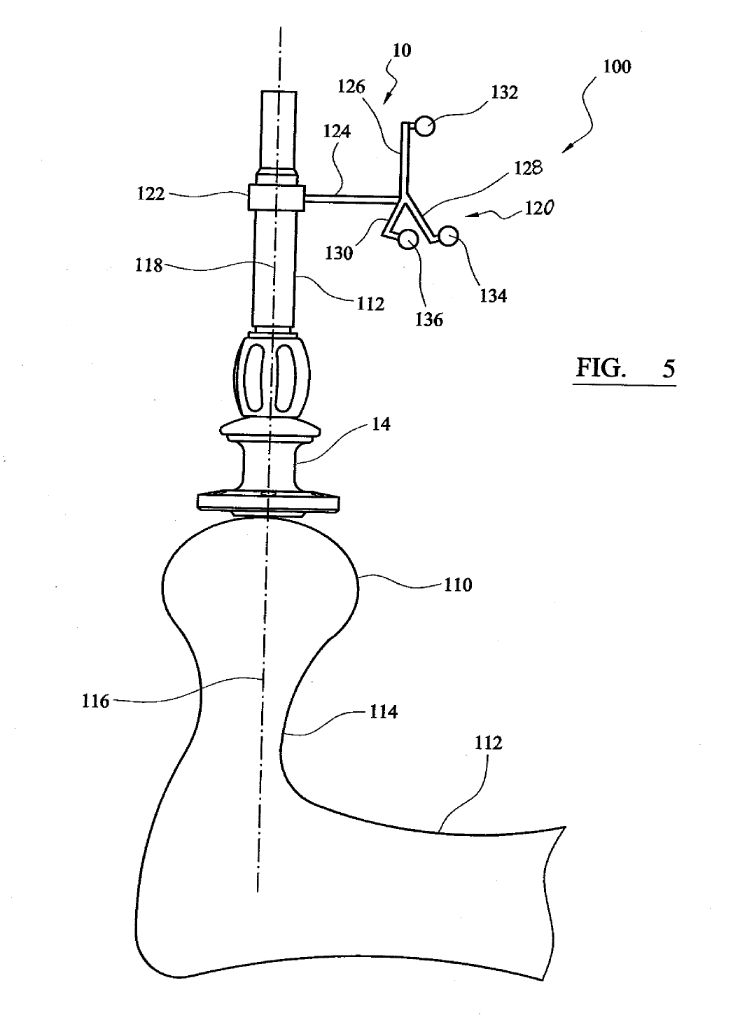

FIG. 3 shows an end view from beneath of the guide shown in FIGS. 1 and 2;

FIG. 4 shows a perspective exploded view of the guide shown in FIGS. 1 to 3;

FIG. 5 shows the guide shown in FIGS. 1 to 4 in use attached to a femoral head;

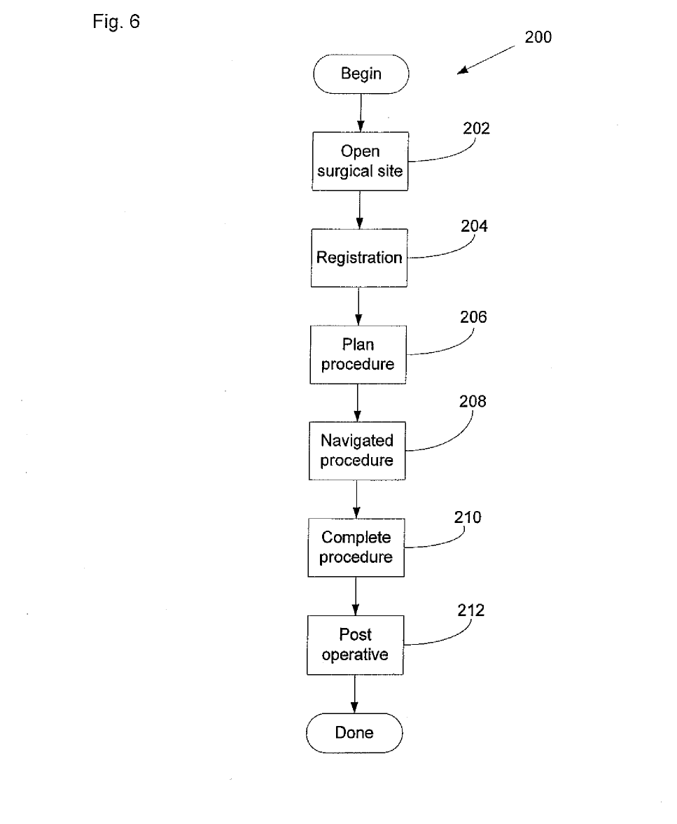

FIG. 6 shows a flowchart illustrating a method of use of the guide according to the invention; and

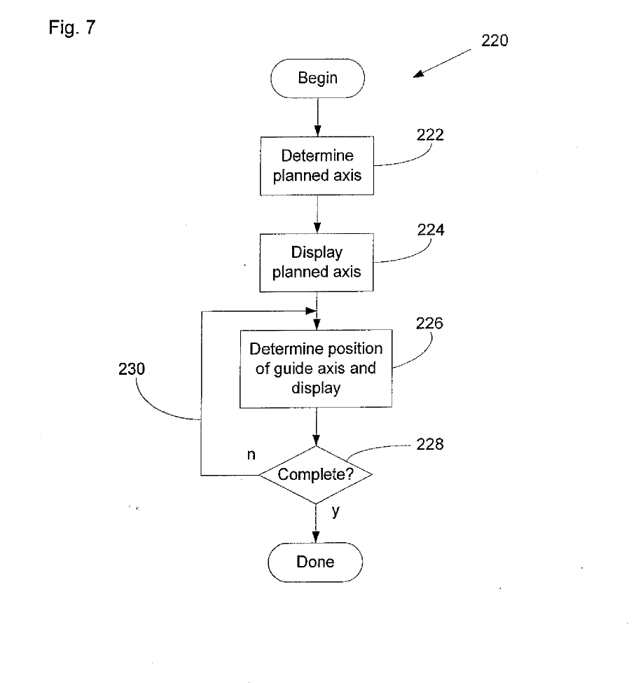

FIG. 7 shows a flowchart illustrating a computer implemented method facilitating use of the guide in a CAS procedure.

Similar items in different Figures share common reference numerals unless indicated otherwise.

In the following a surgical guide will be described in the context of its use in an orthopaedic CAS procedure. However, this is by way of example only and use of the guide is not limited to CAS nor orthopaedic procedures. Rather, the guide can be used in any procedure in which it is helpful to guide an instrument or tool along a preferred axis or direction toward any body part, including bone and soft tissue.

With reference to FIGS. 1 to 4 there is shown a guide 10 according to an embodiment of the invention. Guide 10 includes a body 12 pivotally connected to a base 14.

Body 12 has a circular cross-section channel 16 passing along a longitudinal axis of the guide so as to define a linear guide axis. Body 12 has a generally cylindrical shape and is rotationally symmetric. Body 12 has an upper portion 18 and a lower portion 20. Upper portion 18 has a generally right circular cylindrical shape and lower portion 20 has a curved shape. Concave portions are provided in the surface of lower part 20 to provide grip to a user in use. A proximal end of body 12 includes a threaded portion on the inner wall 24 which in use can engage with a threaded portion of a device 26.

A distal part of the inner surface of body 12 includes as further threaded portion 28 for receiving a mating threaded portion of a stem 30.

Base 14 has a generally circular symmetric shape about its longitudinal axis. Base 14 has a generally annular construction with a cavity 32 therein. A proximal part of base 14 includes a substantially annular channel 34 defined by an outer wall 36 and inner wall 38. A rubber O-ring 40 is located in channel 34.

Toward a distal end of base 14, a skirt 42 extends away from and slightly downwardly from body 14. Six equi-angularly spaced apertures 44 are provided in skirt 42 for receiving a fixing so as to securely fasten guide 10 to a body part.

A distal most part 46 of base 14 provides a fitting which engages the surface of a body part when the guide is mounted on the body part in use. The footing 46 is in the form of a circular rim extending around the central longitudinal axis of the base. Footing 46 defines a plane perpendicular to the longitudinal axis of base 14. A centre of motion about which the guide axis can pivot exists at the centre of that plane, as will be described in greater detail below.

Recessed within base 14 is a formation 48 having an outer surface corresponding to a part of the surface of a sphere. Curved formation 48 provides a socket part of a ball and socket joint. Guide 10 also includes a stem part 30. Stem 30 also has a generally cylindrically shape and is generally rotationally symmetric. A threaded portion 50 is provided at a proximal end of stem 30. A circular cross-section channel 52 extends along the length of stem 30. A formation 54 is provided at a distal end of stem 30. Formation 54 has a curved outer surface generally in the shape of a part of a sphere. As illustrated best in FIG. 2, when guide 10 is assembled, stem 30 is connected to body 12 with curve formation 54 being located in socket 48. Curved formation 54 provides a part of a ball such that formations 54 and 48 provide a ball and socket type joint about which body 12 and stem 30 can pivot.

Element 56 has the general form of a concave disk and has a central aperture 58 therein. Element 56 has a curved underside bearing surface 60. Bearing surface 60 has the shape of a part of a sphere with a radius of curvature centred on the centre of motion at the centre of footing 46. Inner wall 36, outer wall 38 and O-ring 40 between them provide a bearing surface of base 14 over which element 56 can move in use. The edge of element 56 around aperture 58 has a flat section and threaded portion 50 of stem 30 as a similar shape such that element 56 cannot rotate relative to stem 30.

Device 26 is in the form of an elongate instrument having an extended shaft 62 with a sharp point 64 at a distal end thereof. A threaded portion 66 is provided toward a proximal end of shaft 62. A head 68 is provided at distal end of device 26. Device 26 can be accepted within channel 16 of body 12 and channel 52 of stem 30.

The components of guide 12 are configured such that the guide axis passing along the centre of channels 16 and 52 passes through the centre of the outermost plane of footing 46 and so that the guide axis can be pivoted about that point while always passing through that point, and while always passing through that point. The curvature of bearing surface 60 constrains motion of the body 12 and stem 30 so that the guide axis always passes through this point, being a centre of motion for pivotal movement of the body and stem of the guide. Hence, the locus of the guide axis in space, is a cone having a tip at the centre of motion.

In use, by unscrewing body 12 slightly from stem 30, element 56 is freed and the guide axis can be pivoted about the centre of motion as guided by element 56 and the partial ball and socket joint provided by formations 54 and 48. When the guide axis is at the correct orientation relative to the base, then by rotating body 12 relative to stem 30, element 56 is compressed against outer wall 36, O-ring 40 and inner wall 38 so as to fix the orientation of the guide axis relative to the base 14.

Although device 26 is shown inserted in the guide channel of the guide in FIGS. 1 to 3, it will be appreciated that in use, the device 26 need not be inserted in the guide.

Use of the guide in an articulate surface replacement (“ASR”) procedure will now be described with reference to FIGS. 5, 6 and 7. However, it will be appreciated that use of the guide is not limited to such a surgical procedure and can be used in any surgical procedure in order to define or determine an axis or direction relative to a body part along which to guide a tool, instrument or other device.

FIG. 5 shows a schematic illustration 100 showing a guide 10 mounted on the femoral head 110 of a femur 112. Femoral head 110 is connected to the proximal part of femur 112 by the femoral neck 114. The femoral neck has an approximate femoral neck axis as illustrated by broken line 116 in FIG. 5. Broken line 118 represents the guide axis.

In FIG. 5, guide 10 has been adapted for use in a computer aided surgery (“CAS”) system by having a marker array 120 affixed thereto. Marker array, also sometimes referred to as a star in the art, includes a clamp 122 and a stem 124 supporting three arms 126, 128, 130. A reflective sphere 132, 134, 136 is attached to the pre-end of each arm. Such a marker array is suitable for use with tracking systems which detect reflected radiation, such as infrared radiation, such as the vector vision system as provided by BrainLab GmbH of Germany.

Other markers can be used, based on other tracking technologies as will be apparent to a person of ordinary skill in the art, for example, ultra sonic or other acoustic markers can be used. Furthermore, a magnetic marker can be used which detects variations in a magnetic field having a known field distribution and which uses three mutually orthogonal sensor coils to determine and transmit position co-ordinates and angular orientation information to a tracking system.

Irrespective of the tracking technology used, the tracking system or CAS system is pre-programmed with information sufficient to identify the marker being tracked and also has data from which the position of the marker relative to the longitudinal axis of the guide and the position from the centre of motion of the guide. Hence, in use, the tracking system monitors the current position of the marker and from that position and orientation information, the CAS system can determine the current position of the guide axis 118 in the reference frame of the tracking system.

FIG. 6 shows a flowchart providing a high level overview of a CAS procedure 200 in which guide 10 can be used. At step 202, the surgeon makes various incisions to gain access to the surgical site.

At step 204, the patient's body parts are registered with the CAS system so that the position of the patient's bones in the reference frame of the tracking system is known.

Various techniques for registering bones with the CAS system can be used and are generally known in the art. In one such method, the CAS system stores a number of generic virtual models of bones. The surgeon then uses a trackable pointer to identify some major anatomical landmarks so that the overall shape and size of the bone can be determined. The system then selects a one of the generic models from a database of such models which most closely matches the shape and size of the patient's actual bone. Then the surgeon collects a net or mesh of points extending over a significant surface area of the patient's actual bone. Using this shape information derived from the tracking system, the generic bone model can then be morphed to more accurately represent the patient's actual bone shape. In this way, the bone is registered with the CAS system. In alternate embodiments, a non-invasive registration procedure can be used prior to opening the surgical site at step 202.

At step 206, the surgeon can use planning software in order to plan the surgical procedure to be carried out. In the present example of articulate surface replacement, a one of the steps in the workflow of the surgeon is to drill a hole substantially along the femoral neck axis which will receive the stem of an orthopaedic femoral head implant. Correct positioning of the femoral head implant is important in order to ensure correct functioning of the replacement hip joint and also to help minimise and reduce wear of the replacement joint. Using the planning software, at step 206, the surgeon can select the appropriate sizes of the femoral head and acetabular cup implants and also determine the appropriate positions and orientations of the implants relative to the patient's femur and pelvis. Typically, such planning software uses previously captured images, e.g. from a CT scan, or X-ray scan, of the patient's pelvis and femur so that the surgeon can plan the intended position and orientation of the implants using an accurate representation of the patient's bones. Although planning is shown as an intra-operative step 206 in FIG. 6, in alternate embodiments of the invention, planning can be carried pre-operatively, before the surgical site has been opened. Planning can also be carried out before the patient has been registered depending on how anatomical data is gathered.

It will be appreciated from the above that the particular order of carrying out the steps shown in FIG. 6 is not necessarily important and the order of the various steps can be varied. However, in general, before the surgeon can position the guide using CAS, the surgeon needs access to anatomical data, a registration transformation matrix (which mathematically defines the transformation between the actual position of the patient and the reference frame of the tracking system), a plan of where the trajectory is and the calculated surface intersection point between the trajectory and the femoral head, which gives the entry point.

Then at step 208, various actions are carried out by the surgeon so as to carry out the hip replacement procedure. Using a CAS system, the implants, instruments, tools and other devices used in the procedure can have markers trackable by the tracking system attached thereto so that the position of the implants, instruments, tools and devices can be navigated by the surgeon using a display screen which guides the surgeon by providing visual indications of the actual current positions of the implants, instruments, tools and devices relative to the patient's body parts and any planned positions and/or orientations.

The entire hip replacement procedure will not be described herein so as not to obscure the present invention. However, various steps which can be carried out using guide 100 of the present invention will be described.

FIG. 7 shows a flowchart illustrating a process 220 carried out by a data processing part of the CAS system so as to provide a display to facilitate the image guided surgery (“IGS”) procedure. At step 222, the CAS system determines the planned axis 116 which the surgeon has previously planned to drill a hole along. The CAS system has access to this information from the planning software and tracks the current position of the bone so as to provide a real time display of the intended axis 116 to the surgeon at step 224.

As the angular position only of guide 10 can be varied, it is important to ensure that the base 14 of guide 10 is accurately positioned on the surface of the femoral head 110 so that its centre of rotation is coincident with the entry point on the surface of the bone.

At step 226, the tracking system detects and tracks the position of the marker 120 and the CAS system generates and displays in real time a graphical indication of the position of the guide axis 118 on the display to the surgeon. Hence the surgeon can use the visual indication of the guide axis and the visual indication of the entry point into the bone so as to guide the correct positioning of the base 14 on the surface of femoral head 110. Once the guide is correctly positioned on the femoral head, then pins, screws or other fixings are introduced into apertures 44 so as to securely fix the guide base to the femoral head and prevent it moving. The surgeon can release device 26 by unscrewing it and then applying a sharp tap to the head 68 of device 26 so as to provide an initial guide pole or indent in the surface of femoral head 110.

The surgeon then slightly unscrews body 12 so that the body becomes slightly loose from the base 14 and the surgeon can then pivot body 12 so as to align guide axis 118 with the intended body part axis 116 that has been guided by the display unit of the IGS system. The CAS system continually tracks and displays a visual indication of the position of guide axis 118 whilst the surgeon manipulates the guide to align it with the body axis, as illustrated by decision box 228 and process flow return line 230. When the surgeon is satisfied that the guide axis 118 and intended body part axis 116 are aligned, then the surgeon simply tightens the guide by rotating body 12. Rotating body 12 draws stem 30 into body 12 thereby applying a compressive force on element 56 as stem 30 is retained in base 14. Hence rotating body 12 causes the guide to lock with the body 12 at a fixed angular orientation relative to base 14. The surgeon can then use guide 10 to guide the drilling of a pilot hole via guide channels 16 and 52 and then drilling a further hole to receive the stem of the femoral head prosthesis.

Returning to FIG. 6, the guide can then be removed and the surgical procedure will then be completed at step 210. Then at step 212, the surgeon can carry out any checks on the performance of the replacement hip joint and finally close the surgical site.

Hence, it will be appreciated that the above described guide has a simple construction and is easy to use as the guide channel can only be pivoted relative to the base, thereby providing ease of operation and a reduced number and complexity of component parts.

The parts of guide 10 can be made from any material typically used for the manufacture of surgical tools or instruments, such as stainless steel or other corrosion resistant alloys. In one embodiment the guide can be made of plastics, in which case the guide can be provided as a disposable instrument.

1. A surgical guide mountable on a body part to guide a device along a guide axis, the guide comprising:
a body having a channel therein defining the guide axis and configured for receiving the device in use;
a base configured to engage a surface of the body part when mounted thereon in use and support the body over an entry point for the body part such that the guide axis passes through the entry point; and
a pivot mechanism attaching the body to the base, wherein the body can move relative to the base only by pivoting, and with at least two pivoting degrees of freedom, and wherein the guide is configured such that the entry point is a center of motion about which the body pivots.

2. The guide of claim 1, further comprising a lock operable to fix the angular position of the body relative to the base.

3. The guide of claim 1, wherein the pivot mechanism comprises a ball-and-socket joint provided by a rotating part and a stationary part.

4. The guide of claim 1, wherein the rotating part and the stationary part between them provide a bearing surface being at least a part of a sphere and a further bearing structure over which the bearing surface can move.

5. The guide of claim 1, wherein the pivot mechanism includes:
an element having a first bearing surface being a part of the surface of a sphere; and
a second bearing surface over which the first bearing surface can move, wherein the radius of curvature of the first bearing surface extends between the first bearing surface and the entry point.

6. The guide of claim 1, wherein the base comprises a plurality of attachment points for securing the guide to the body part.

7. The guide of claim 1, wherein the guide includes a stem, the stem being attached to the body and passing into the base, the stem having a further channel being an extension of the channel and being co-linear with the guide axis.

8. The guide of claim 2, wherein the guide includes a stem, the stem being attached to the body and passing into, and being retained by, the base, and wherein the body and stem co-operate to provide the lock.

9. The guide of claim 1, further comprising a marker attached to the body, wherein the marker is trackable by a tracking system to determine the position of the guide axis in a co-ordinate system of the tracking system.

10. The guide of claim 1, wherein the guide has a longitudinal axis and each part of the guide is rotationally symmetric about the longitudinal axis.

11. A method for determining an axis along which a device is to be directed toward a body part, the method comprising:
mounting a guide device on the body part over an entry point on the surface of the body part, the guide having a base and a body part having a channel for receiving the device along a guide axis, the body being movable relative to the base by pivoting only; and
only pivoting the body of the guide to vary the orientation of the guide axis while passing through the entry point until the guide axis is substantially coincidental with the axis.

12. A method for determining an axis along which a device is to be directed toward a body part, the method comprising:
determining the orientation of the axis and an entry point through which the axis passes on the surface of a body part in a co-ordinate system of a tracking system;
tracking the position of a guide in the co-ordinate system, the guide having a base and a body part, the body part having a channel for receiving the device along a guide axis, the body being movable relative to the base by pivoting only; and
displaying a graphical indication of the position of the guide axis relative to the axis and/or the position of the guide axis relative to the entry point.
